(19) United States
(12) Patent Application Publication
(10) Pub. No.: US 2026/0340790 A1
(43) Pub. Date: Oct. 29, 2026

(54) GENERATING SYNTHETIC MEDICAL IMAGES, FEATURE DATA FOR TRAINING IMAGE SEGMENTATION, AND INPAINTED MEDICAL IMAGES USING GENERATIVE MODELS

(71) Applicant: Mayo Foundation for Medical Education and Research, Rochester, MN (US)

(72) Inventors: Cody C. Wyles, Rochester, MN (US); Pouria Rouzrokh, Rochester, MN (US); Bardia Khosravi, Rochester, MN (US); Michael J. Taunton, Rochester, MN (US); Bradley J. Erickson, Rochester, MN (US)

(21) Appl. No.: 19/111,683

(22) PCT Filed: Sep. 14, 2023
(86) PCT No.: PCT/US2023/074166

§ 371 Date: Mar. 13, 2025

Related U.S. Application Data
(60) Provisional application No. 63/406,375, filed on Sep. 14, 2022.

(51) Int. Cl. G06T 5/77; G06T 5/60; G16H 10/60; G16H 30/40

(52) U.S. Cl. CPC G06T 5/77; G06T 5/60; G16H 10/60; G16H 30/40

(57) ABSTRACT

Generative models (e.g., a denoising diffusion probabilistic model (“DDPM”) or other suitable generative model) are used to create synthetic medical images (e.g., synthetic digital radiographic images), feature data useful as a training data set for training an image segmentation model, inpainted medical images that depict a predicted postoperative outcome for a patient, and/or deidentified medical images in which radiographic markers have been removed.

### BACKGROUND

Deep learning is a rapidly expanding branch of artificial intelligence research in medicine with the promise of enhancing patient care. Deep learning models are generated by training neural networks (or other deep learning models) on large datasets of imaging, text, or tabular data. Deep learning models are the current state-of-the-art tools for performing several tasks with medical imaging data, including classification of patients' diagnoses or prognoses, identification and segmentation of objects of interest, and the generation of synthetic data.

For instance, image segmentation is a routine part of most computer-aided medical image analysis pipelines. As a downside, training segmentation models usually need a lot of training data, annotated by experts, which is time-consuming and can be costly. Furthermore, many anatomical landmarks may be challenging to discern in medical images, making the annotation of accurate training data sets even more complex.

### SUMMARY OF THE DISCLOSURE

The present disclosure provides a method for generating an inpainted medical image. The method includes accessing a preoperative medical image with a computer system, where the preoperative medical image depicting patient anatomy, and accessing a generative model with the computer system, where the generative model has been trained on training data that includes preoperative and postoperative medical images. The preoperative medical image is input to the generative model using the computer system, generating an output as an inpainted medical image that depicts a predicted postoperative outcome for the patient.

It is another aspect of the present disclosure to provide a method for generating synthetic medical images. The method includes accessing medical image data with a computer system, where the medical image data depict patient anatomy. A generative model is also accessed with the computer system, where the generative model has been trained on training data that include medical images that are consistent with the medical image data. The medical image data are then input to the generative model using the computer system, generating an output as synthetic medical image data having an augmented feature.

It is still another aspect of the present disclosure to provide a method generating training data for use with training a machine learning model to generate a segmented medical image. The method includes accessing a pre-trained generative model with a computer system, where the pre-trained generative model has been trained on training data that includes medical images. Medical image data are also accessed with the computer system, where the medical image data are consistent with the training data. The medical image data are input to the generative model using the computer system. Feature data are extracted from the generative model while the generative model is processing the medical image data. An image segmentation model is then trained on the feature data extracted from the generative model as a training data set.

It is yet another aspect of the present disclosure to provide a method for generating a deidentified medical image. The method includes accessing a medical image with a computer system, where the medical image depicts a subject and at least one radiographic marker. A machine learning model is also accessed with the computer system, where the machine learning model has been trained on training data to identify and remove radiographic markers in medical image data. The medical image is input to the machine learning model using the computer system, generating a deidentified medical image as an output. The deidentified medical image has the at least one radiographic marker removed therefrom. The deidentified medical image may then be displayed to a user via the computer system.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1A shows example synthetic digital radiographic images generated using one example implementation of a generative model described in the present disclosure.

FIG. 1B shows example synthetic super-resolution digital radiographic images generated using one example implementation of a generative model described in the present disclosure.

FIG. 2 illustrates examples of conditionally generated synthetic images with their input characteristics. Underlined words are variables that are fed to the DDPM as conditions.

FIG. 3 illustrates an example generative model and a process for extracting features from intermediate blocks of the generative model to be used as training data for training an image segmentation model.

FIG. 4A shows example digital radiographic images segmented using an image segmentation model trained on feature data extracted from a generative model.

FIG. 4B shows an example of a segmentation model compared with the ground truth.

FIG. 5 illustrates an example training process for training a DDPM to generate inpainted medical images according to some embodiments described in the present disclosure.

FIG. 6 illustrates an example process for generating inpainted medical images using a DDPM-based generative model according to some embodiments described in the present disclosure.

FIG. 7A shows example images used for training a DDPM to generate inpainted medical images.

FIG. 7B shows example post operative images used for training a DDPM to generate inpainted medical images.

FIG. 7C shows example inpainted images generated using a trained DDPM according to some embodiments described in the present disclosure.

FIG. 8 illustrates variations of synthetic postoperative radiographs generated by the disclosed generative models (referred to as THA-Net in the illustrated example) for a single preoperative radiograph with different prespecified femoral component types in each generation. The generations are done in the Hardware-aware mode, but the choice of acetabular component was not prespecified to the model.

FIG. 9 illustrates variations of synthetic postoperative radiographs generated by THA-Net for a single preoperative radiograph within the Automated mode. THA-Net has used different femoral component types for generating different synthetic postoperative radiographs, a finding that may be related to the non-standard preoperative radiograph fed to the model during generation (top left hand side).

FIG. 10 illustrates variations of synthetic postoperative radiographs generated by THA-Net for a single preoperative radiograph within the Automated mode. THA-Net has used the same femoral component type for generating different synthetic postoperative radiographs, a finding that indicates the certainty of the model in its hardware choices for performing the templating task.

FIG. 11 illustrates visualizations of images from reviewer evaluation pools with highest and lowest total validity ratings in Hardware-aware and Automated generations. Each reviewer rated the validity of radiographs on a 10-point Likert scale, so the maximum possible rating is 20. The ratings of real counterparts of generated radiographs are also provided for comparison purposes.

FIGS. 12A-12C illustrate examples of locating radiographic markers in medical images to generate deidentified medical images. FIG. 12A shows marker detection on pelvic radiographs. Note that the marker areas were obfuscated to ensure patient privacy. FIGS. 12B and 12C show the results of the localizer model's performance on out-of-domain chest radiographs before (FIG. 12B) and after (FIG. 12C) fine tuning. The red boxes are zoomed-in versions of the marker area that was detected.

FIG. 13 illustrates an example workflow for selectively removing radiographic markers from a medical image. In this example, non-sensitive radiographic markers (e.g., laterality markers) are retained while markers that contain sensitive information (e.g., PHI or otherwise) are removed.

FIG. 14 is a flowchart setting forth the steps of an example method for generating deidentified medical images using a suitably trained machine learning model.

FIG. 15 is a flowchart setting forth the steps of an example method for training a machine learning model to generate deidentified medical images.

FIG. 16 is a block diagram of an example system for medical image generative modelling, which may be used to generate synthetic medical images, extract feature data for training an image segmentation model, generating inpainted medical images, and/or generating deidentified medical images as described in the present disclosure.

FIG. 17 is a block diagram of example components that can implement the system of FIG. 16.

### DETAILED DESCRIPTION

Described here are systems and methods for using generative models (e.g., a denoising diffusion probabilistic model (“DDPM”) or other suitable generative model) in various medical imaging applications. For instance, the systems and methods described in the present disclosure can be used to create synthetic medical images (e.g., synthetic digital radiographic images), which may include synthetic medical images with a higher spatial resolution than the input medical image data, synthetic medical images with lower noise than the input medical image data, synthetic medical images with otherwise improved image quality, and so on. The systems and methods described in the present disclosure can also be used to generate segmented medical image data, which may include segmented medical images. In still other instances, the systems and methods described in the present disclosure can be used to generate inpainted medical images, in which a potential surgical outcome is depicted in the inpainted image. For example, the inpainted medical images may depict an ideal, or otherwise preferable, orthopedic implant for a particular patient. In this way, the inpainted medical images can provide a prediction of ideal, or otherwise preferable, postoperative medical images, which in a non-limiting example may include total hip arthroplasty postoperative digital radiographic images. Additionally or alternatively, the systems and methods described in the present disclosure can be used to generate deidentified medical images, including medical images in which radiographic markers or other protected health information are located and removed (or otherwise marked for redaction).

As one example, generative models are trained and used to create synthetic medical images from input medical image data. The medical image data can include digital radiographic images, fluoroscopy images, computed tomography (“CT”) images, magnetic resonance images, and so on. Thus, in one example, a generative model can be trained and used to create synthetic digital radiographic images from medical image data that are input to the generative model. As noted above, the synthetic medical images can include an improvement in one or more image quality metrics. For instance, the synthetic medical images may have an improved spatial resolution (e.g., super-resolution), an improved signal-to-noise ratio (e.g., reduced image noise), and so on.

As another example, the synthetic medical images can have different properties than the input medical image data, including images with different spatial transformations (e.g., translations, rotations, etc.), and so on. For instance, a digital radiographic image may depict patient anatomy that is tilted and/or rotated out of a desired anatomical plane. In these instances, the generative model can receive the skewed image as an input and can generate a synthetic image that depicts the patient anatomy in a desired anatomical plane as an output. For example, the generative model can convert a tilted image into a standard projection. As another example, the generative model can generate a three-dimensional image (e.g., which may represent a three-dimensional reconstruction of patient anatomy, such as a three-dimension reconstruction of one or more bones) from a two-dimension medical image, such as a two-dimensional digital radiographic image.

In some instances, the synthetic medical images may include medical images that are representative of a patient population based on demographic or other features of the patient population. For example, in some instances the synthetic medical images may be representative of a particular biological sex group, ethnic group, age group, or combinations thereof.

It is an aspect of the present disclosure that the intermediate layers of a generative model can include feature data that, when extracted from the intermediate layers of the generative model, can be used as training data for other machine learning models, such as a machine learning model used for image segmentation.

As another example, generative models are trained and used to generate inpainted medical images from input medical image data. Inpainting, in the context of deep learning, is a technique where a generative model is conditioned to fill in missing or corrupted parts of an image. This approach allows the model to generate plausible content that seamlessly fits with the uncorrupted parts of the image. As with other implementations, the medical image data can include digital radiographic images, fluoroscopy images, computed tomography (“CT”) images, magnetic resonance images, and so on. The inpainted medical images can be generated by inputting medical image data to a generative model. In some instances, additional data can also be input to the generative model, such as patient health data. The patient health data may include patient age, patient biological sex, patient body mass index (“BMI”), patient indication for surgery, patient choice for a surgical approach (e.g., anterior approach, posterior approach) pertaining to the surgical procedure for the patient, a patient or surgeon choice for a particular type of orthopedic implant, other patient health data, or combinations thereof. As noted above, the inpainted medical images generally depict a predicted or otherwise potential surgical outcome for a patient.

As one example, the inpainted medical image can depict a predicted or otherwise potential outcome of an orthopedic procedure. For instance, the inpainted medical image may depict an orthopedic implant (e.g., a hip implant, a knee implant, a shoulder implant, spinal fixation implants, other bone fixation implants, and so on). Advantageously, the orthopedic implant depicted in the inpainted medical image can represent an ideal, or otherwise preferable, orthopedic implant for the specific patient. For instance, the orthopedic implant depicted in the inpainted medical image can represent the orthopedic implant most likely to minimize the risk of post operative complications for the specific patient based on the patient's anatomy and/or other relevant factors (e.g., features in the patient health data). In this way, the systems and methods described in the present disclosure are capable of generating an inpainted image that not only depicts a positioning of an optimized orthopedic implant in a particular patient, but represents the selection of that optimized orthopedic implant. That is, the generative model is capable of selecting an ideal, or otherwise preferable, orthopedic implant by way of generating the inpainted medical image. As such, the inpainted medical image can be generated as part of a pre-procedure report for an orthopedic surgeon in order to assist with the selection of an orthopedic implant for a patient, as well as for planning the related surgical procedure.

As yet another example, the inpainted medical image can depict a branding of the hardware to be used for an orthopedic, or other, surgical procedure. For instance, the inpainted medical image can pre-specify, as an input to the generative model, the branding of the hardware (e.g., cup or stem) that is to be used for the orthopedic procedure. In this way, the patient and/or surgeon can specify a particular orthopedic implant and visualize how that particular implant will look within the patient's particular anatomy.

In addition to orthopedic implants and procedures, the inpainted medical images can also depict other surgical implant outcomes. For instance, the inpainted medical images may depict ideal, or otherwise preferable, implant selection and positioning for other implanted medical devices, such as neurostimulators (e.g., spinal cord stimulators, deep brain stimulators, cranial nerve stimulators), implantable cardiac electrical devices (e.g., pacemakers, cardioverter-defibrillators (“ICDs”)), brachytherapy seeds, and so on.

Additionally or alternatively, the inpainted medical images can depict other surgical outcomes, such as musculoskeletal reconstructions. For instance, a patient who suffered severe trauma to a limb may have both muscular and skeletal damage to the limb. One or more inpainted medical images can be generated to depict an ideal, or otherwise preferable or predicted, reconstruction of the patient's normal musculoskeletal anatomy. For example, one or more inpainted medical images may depict reconstructed muscular anatomy (e.g., an inpainted magnetic resonance image), reconstructed skeletal anatomy (e.g., an inpainted digital radiographic and/or x-ray CT image), or the like.

As noted above, the systems and method described in the present disclosure implement one or more generative models. In general, a generative model includes a model that is capable of describing how a dataset is generated in terms of a statistical and/or probabilistic model. The generative model can be sampled (e.g., by inputting a new data set to the generative model) in order to generate new data that is consistent with, or plausibly derivable from, the modeled data set. Generative models may be trained or otherwise constructed using machine learning and/or deep learning. As such, the generative models may be referred to as machine learning models, deep learning models, or the like. The generative models described in the present disclosure can be trained using unsupervised learning techniques, supervised learning techniques, or combinations of both, depending on the desired output of the generative model.

As a non-limiting example, the generative models implemented in the present disclosure can include a generative adversarial network (“GAN”) (e.g., a ProGAN model, a StyleGAN model, a BigGAN model, and so on), a variational autoencoder (“VAE”), a denoising diffusion probabilistic model (“DDPM”), or the like. GANs have high inference speed and can generate high quality output samples, but may be limited in the diversity of outputs that they can generate. VAEs have high inference speed and output diversity, but may be limited by the quality of samples generated. DDPMs can produce high quality and diversity outputs, but may be limited by longer inference times. Thus, while many different types of generative models may be used when implementing the systems and methods described in the present disclosure, in some preferred embodiments a DDPM-based generative model may be used.

In general, a DDPM is a parameterized Markov chain trained using variational inference to produce samples matching the data after finite time. Transitions of this chain can be learned to reverse a diffusion process, which is a Markov chain that gradually adds noise to the data in the opposite direction of sampling until signal is destroyed. When the diffusion includes small amounts of Gaussian noise, it is sufficient to set the sampling chain transitions to conditional Gaussians, too, thereby allowing for a particularly simple neural network parameterization.

As is the case with other deep learning models, DDPMs are trained by gradually encountering a prepared set of images, known as training data. In some instances, a DDPM can be trained using unsupervised learning techniques, as noted above. However, DDPMs can also be utilized for supervised or conditional training, as discussed below. In the context of deep learning, conditioning refers to the practice of guiding the output of a model based on certain variables or data. For generative models, including DDPMs, conditioning may involve generating data with specific characteristics, based on provided context or input parameters. One approach for conditioning a DDPM model on user input data include classifier-free conditioning, thereby eliminating the need for a pre-trained classifier during training. This can significantly enhance the feasibility of training conditional DDPM.

As a non-limiting example, to train an unsupervised DDPM model, each image in the training data is first converted to a “noisy” version by adding a certain quantity of Gaussian noise (e.g., noise intensity originates from t; 1<t<T) using Markov chain theory (called forward diffusion). If T is sufficiently large, the image noise at the final step (t=T) should be isotropic random noise. When all training data is converted to noisy versions, as described previously, the DDPM will then be trained to denoise the noisier copies of the images to their less noisy equivalents, and ultimately to the original images. This is referred to as reverse diffusion. Once the DDPM has been successfully trained, it can be implemented to transform random noise data into a meaningful image similar to, consistent with, or otherwise plausibly derivable from the data it was trained on. In other words, a trained DDPM can accept any random noise as input and is not limited to the noisy version of the imaging data present in the training set.

An example process for constructing a DDPM is as follows. Given a data distribution x₀~q(x₀), a forward noising process (q) that produces latent x₁ through x_{T} can be defined by adding Gaussian noise at time t with variance βₜ∈(0,1) as follows:

$$q ( {x}_{1} , … , {x}_{T} \vert {x}_{0} ) := {\prod }_{t = 1}^{T} q ( {x}_{t} \vert {x}_{t - 1} ) \qquad ( 1 )$$

$$q ( {x}_{t} \vert {x}_{t - 1} ) := 𝒩 ( {x}_{i} ; \sqrt{1 - {β}_{i}} {x}_{t - 1} , {β}_{t} I ) \qquad ( 2 )$$

Given sufficiently large T and a well behaved schedule of βₜ, the latent x_{T} is nearly an isotropic Gaussian Distribution. Thus, if the exact reverse distribution q(xₜ₋₁|xₜ) is known, x_{T}~N(0,I) can be sampled and the process ran in reverse to get a sample from q(x₀). Because q(xₜ₋₁|xₜ) depends on the entire data distribution, it can be approximated using a neural network as follows:

p_{θ}(xₜ₋₁|x₁):=N(xₜ₋₁:μ_{θ}(xₜ,t),Σ_{θ}(xₜ,t)) (3)

The noising process defined in Equation 2 allows for the sampling of an arbitrary step of the noised latent directly, conditioned on the input x₀. With αₜ: =1-βₜ and

$$\overline{{α}_{t}} := {\prod }_{s = 1}^{t} {α}_{s} ,$$

the marginal can be written as:

q(xₜ|x₀)=N(xₜ;\sqrt{α̅ₜ}x₀,(1−α̅ₜ)I) (8)

xₜ=\sqrt{α̅ₜ}x₀+1−\sqrt{α̅ₜ}ϵ (9)

Using Bayes Theorem, the posterior q(xₜ₋₁|xₜ,x₀) can be calculated in terms of β̅ₜ and μₜ (xₜ,x₀), which can be defined as follows:

$${\tilde{β}}_{t} := \frac{1 - {\overline{α}}_{t - 1}}{1 - {\overline{α}}_{t}} {β}_{t} \qquad ( 10 )$$

$${\overline{μ}}_{t} ( {x}_{t} , {x}_{0} ) := \frac{\sqrt{{\overline{α}}_{t - 1}} {β}_{i}}{1 - {\overline{α}}_{t}} {x}_{0} + \frac{\sqrt{{α}_{t}} ( 1 - {\overline{α}}_{t - 1} )}{1 - {\overline{α}}_{t - 1}} {x}_{t} \qquad ( 11 )$$

$$q ( {x}_{t - 1} \vert {x}_{t} , {x}_{0} ) = 𝒩 ( {x}_{t - 1} : \tilde{μ} ( {x}_{t} , {x}_{0} ) , {\tilde{β}}_{t} I ) \qquad ( 12 )$$

There are many different ways to parameterize μ_{θ}(xₜ,t) in the prior. One example option is to predict μ_{θ}(xₜ,t) directly with a neural network. Alternatively, the network could predict x₀, and this output could be used in Equation 11 to produce μ_{θ}(xₜ,t). The network could also predict the noise and use Equations 9 and 11 to derive:

$${μ}_{θ} ( {x}_{t} , t ) = \frac{1}{\sqrt{{α}_{t}}} ( {x}_{t} - \frac{{β}_{t}}{\sqrt{1 - {\overline{α}}_{t}}} {ϵ}_{θ} ( {x}_{t} , t ) ) \qquad ( 13 )$$

As one example, the noise can be predicted and the model can be trained using mean squared error loss:

Lₛᵢₘₚₗₑ=E_{t,x}_{_{α}}_{,ϵ}[∥ϵ−ϵ_{θ}(xₜ,t)∥²] (14)

Recent advances in deep learning have pushed the boundaries of image generation. It is an aspect of the present disclosure that a DDPM, or other generative model, can be used in an end-to-end pipeline to efficiently create high-resolution synthetic medical images to facilitate data augmentation techniques. As a non-limiting example, the synthetic medical images can include synthetic digital radiographic images depicting the pelvis or other anatomical region-of-interest. Additionally or alternatively, the synthetic medical images can include synthetic fluoroscopy images, synthetic CT images, synthetic magnetic resonance images, synthetic ultrasound images, and the like.

In an example implementation, a set of 37,427 native pelvis radiographs were gathered from an institutional registry. A DDPM with a UNet variant architecture and a cosine noise schedule was used with a total of T=1000 steps. To train this model, every image in a batch was converted to a “noisy” version by adding a specific amount of noise (noise intensity comes from t; 1<t<T) based on Markov chain theory (called forward diffusion). If the Tis large enough the noisy image at the last step (t=T) is isotropic random noise. The DDPM tries to denoise the image to a less noisy version (with the noise level of t−1) through a process called reverse diffusion. The model was trained using MSE loss to compare the denoised image with what it should have looked like.

During inference, the initial data was pure random noise and the generative model denoised it T times to generate a clear image. A similar DDPM model was utilized to convert small synthetic images to 1024×1024 pixels. The latter model was conditioned with the small image as a second channel. Example hyperparameter values and settings for the image generator DDPM and super-resolution DDPM are listed in Table 1.

TABLE 1

| | Image | Super |
|---|---|---|
| Hyperparameter | Generator DDPM | Resolution DDPM |
| Input shape | 1 × 256 × 256 | 2 × 1024 × 1024 |
| Noise Schedule | cosine | cosine |
| Total T Steps | 1000 | 1000 |
| Attention Resolution | 32, 16, 8 | 32 |
| UNet Channels | 128 | 64 |
| UNet Channel Multipliers | 1, 1, 2, 2, 4, 4 | 1, 1, 2, 2, 4, 4 |
| Number of Residual Blocks | 2 | 2 |
| Number of Attention Heads | 4 | 4 |

An example of the image generation model was trained in 49 hours after seeing 25,000,000 images. An example of the super-resolution model was trained after seeing 500,000 images in 71 hours. The inference time for creating one thousand 1024×1024 images was 26 minutes. The Fréchet inception distance (“FID”) 5K for the generated images was 8.2. For comparison, training a StyleGan to create high-resolution images on the same hardware took 15 days.

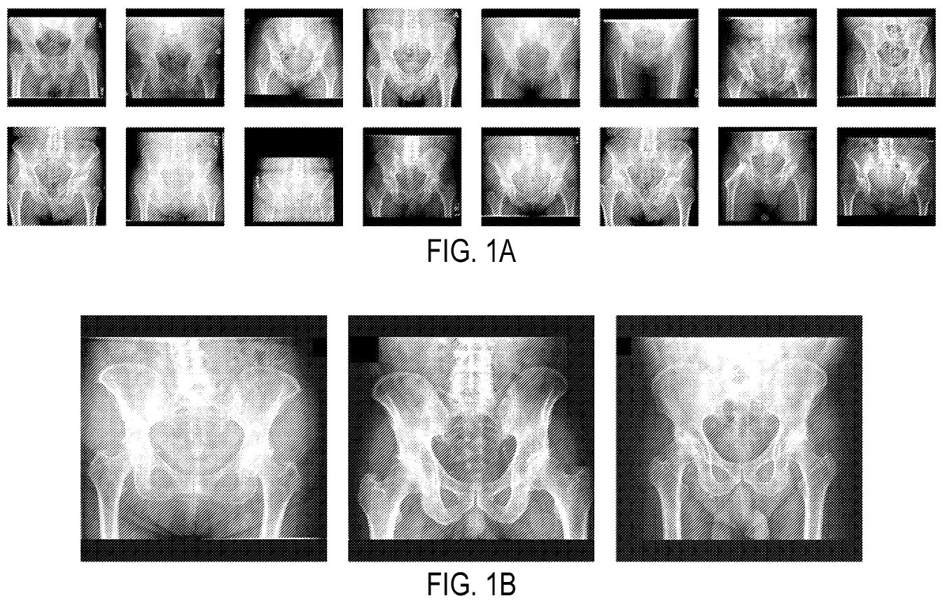

Example images generated using the image generation model is shown in FIG. 1A, and example images generated using the super-resolution model are shown in FIG. 1B. In some implementations, higher-resolution images can be generated by cascading two DDPMs to initially create images in a lower resolution and then increasing their resolution.

Although the example models described above were trained to generate lower and/or higher resolution images for use in data augmentation techniques, synthetic images with other features can also be generated, as discussed above. For example, synthetic medical images with reduced (or increased) noise, synthetic medical images representative of a patient demographic feature (e.g., patient age, patient biological sex, patient ethnicity), and so on, can also be generated. These synthetic images can be used for data augmentation techniques, and can also be used to identify and reduce biases in other machine learning models. For instance, generative models and other machine learning models can reflect the biases in the datasets on which they are trained. As many datasets used for training machine learning models can be very large, it can be difficult to remove these biases, especially when images are unlabeled. Advantageously, being able to generate synthetic medical images with particular features that are representative of a particular model bias can be useful for retraining or otherwise updating a biased machine learning model in order to remove or otherwise reduce those biases.

As a non-limiting example, racial biases can be identified and reduced. In an example study, a DDPM was trained on a training set including 480,407 pelvic radiographs from 15,127 unique patients. Expert evaluators identified six characteristics that were systematically and consistently different between races. The group found that African American patients, when compared to White patients, demonstrated decreased inter-acetabular distance (GAC: 0.83; p-value <0.001); higher degree of osteoarthritis (GAC: 0.82; p-value <0.001); more elliptical obturator foramen (GAC: 0.80; p-value <0.001); a decreased femoral neck-shaft angle (GAC: 0.76; p-value <0.001); elongated pelvis ring (GAC: 0.61; p-value <0.001); and increased femoral metaphyseal cortical thickness (GAC: 0.60; p-value <0.001). Understanding disparities in large medical imaging datasets is crucial, as these can lead to biased downstream models. Generative models can be used to explain complex relationships in the underlying data. Using the systems and methods described in the present disclosure, a dataset explainability approach using generative models is provided to understand racial differences and disparities in a large imaging registry. As generative models are being used to create synthetic data that supplements real images, it is advantageous to understand these biases and control for them during the generation process.

As described above, the deep learning model that is used for the reverse diffusion process may have a U-Net like architecture that gets a noisy version of an image as the input and returns a less noisy version of the same image. Different from generic U-Net models that are used in biomedical image segmentation, the DDPM has several advantageous characteristics, including several residual blocks in each layer; self-attention modules for improved image quality; and embedded timestep numbers along with a conditioning vector that contains class information. As the model is aware of image attributes (through the condition vector), images with specific characteristics can be created by passing the desired classes to the model during inference. As a non-limiting example, the model architecture may include: (1) six levels with depths of 32, 32, 64, 64, 128, 128 and two residual blocks per level; (2) attention blocks at image resolutions of 322, 162, and 82; and (3) an embedding dimension of 128.

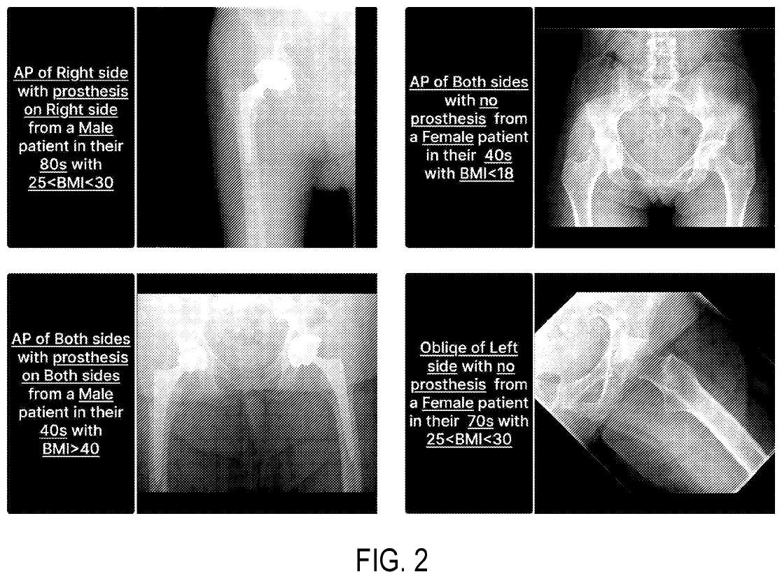

To make the model conditional on demographic and imaging factors, a classifier-free guidance method may be used. Unlike other methods, classifier-free guidance does not require training a separate classifier to make DDPMs conditional. Instead, it uses a learned embedding of null classes (i.e., unknown classes) and randomly chooses a subset of images during training to replace their class embedding with this learned null embedding. FIG. 2 illustrates examples of synthetic images generated using a conditioned DDPM model and their corresponding attributes. In these examples, synthetic images are generated based on user input conditions for the output medical images, including view (e.g., AP or right side, AP of both sides, oblique of left side), presence of implant or prosthesis, patient sex, patient age, and patient BMI. Other conditions can be used, such as patient race or other demographic data for the patient.

Segmentation is a routine part of most computer-aided medical image analysis pipelines. As a downside, training segmentation models usually requires a large volume of training data, annotated by experts, which is time-consuming and can be costly to collect.

It is an aspect of the present disclosure that the described generative models can include feature data that, when extracted, can be used as training data for other machine learning models, such as segmentation models. For instance, a DDPM using a UNet, or other neural network, architecture can include intermediate blocks or layers, from which different feature data can be extracted and used to train a segmentation model with very limited training data.

In an example implementation, a pre-trained DDPM model that generated high fidelity 256×256 pixel anteroposterior pelvis radiographs was used. The hyperparameters for this example model are described in Table 2 below.

TABLE 2

| Hyperparameter | Image Generator DDPM |
|---|---|
| Input shape | 1 × 256 × 256 |
| Noise Schedule | cosine |
| Total T Steps | 1000 |
| Attention Resolution | 32, 16, 8 |
| UNet Channels | 128 |
| UNet Channel Multipliers | 1, 1, 2, 2, 4, 4 |
| Number of Residual Blocks | 2 |
| Number of Attention Heads | 4 |

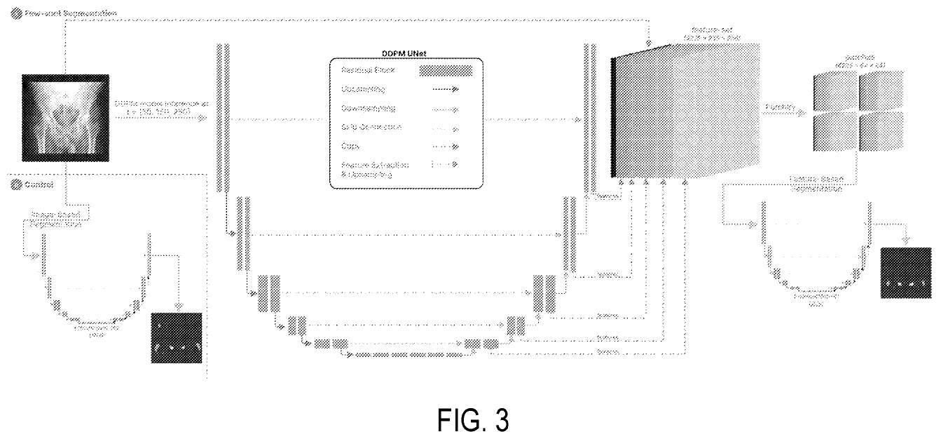

Three anatomical landmarks (greater trochanter [GT], lesser trochanter [LT], and obturator foramen [OF]) were annotated on 30 real-patient digital radiographs, 20 of which were used for training and 10 for validation. To extract features, each image was passed through the pre-trained DDPM at three time steps of t={50, 150, 250} and for each pass, features from blocks b={5, 6, 7, 8, 12} were extracted and upsampled to 256×256 pixels. The features were concatenated with the real image to form an image with 4225 channels and a size of 256×256 pixels (referred to as a feature-set). The feature-set was broken into random 32×32 pixel patches to increase training data. The patches were fed to a small UNet with a depth of two and feature channels of 32 and 64; 2.5 million parameters. To compare the performance, the original images were also used to train a UNet with an EfficinetNet-BO backbone (pre-trained on ImageNet) with 6.5 million parameters. Both models were trained for 500 epochs and the best validation loss was used for model selection. An example of the feature extraction process is illustrated in FIG. 3.

The small UNet trained on the 20 feature-sets achieved a dice score of 0.91, 0.86, and 0.60 for GT, OF, and LT segmentation, respectively. The larger model trained on images alone, only achieved dice scores of 0.56, 0.49, and 0.29 on the three respective classes of GT, OF, and LT.

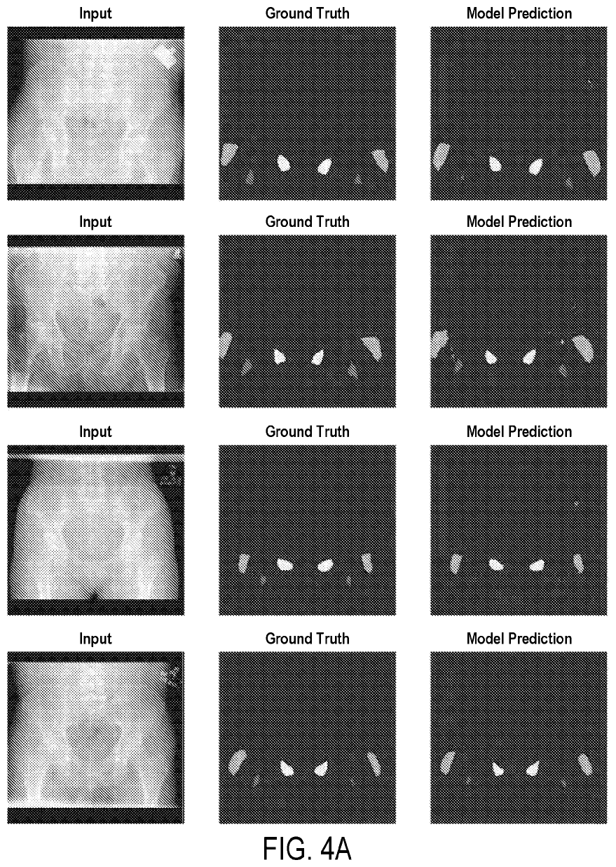

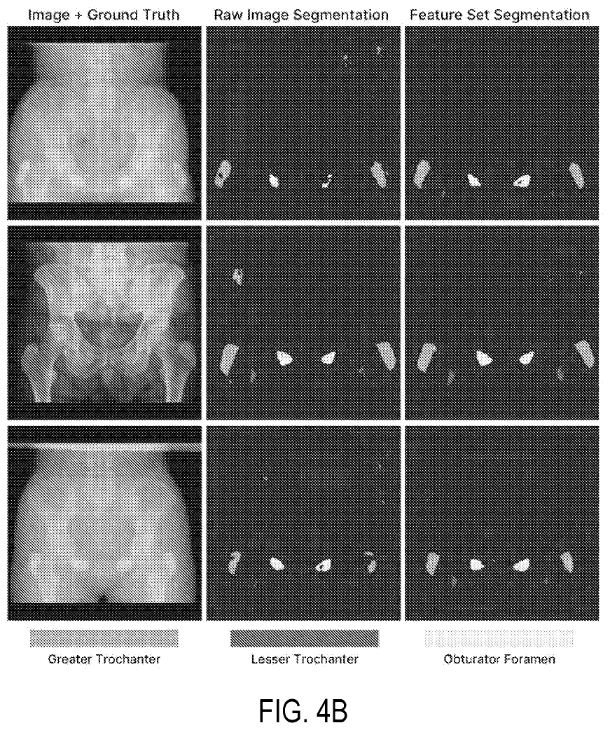

Examples of segmented medical image data generated using a segmentation model trained on feature data extracted from the DDPM generative model are shown in FIG. 4A, and a comparison of the segmentation model with ground truth is shown in FIG. 4B. As noted, the segmented image features include greater trochanter, lesser trochanter, and obturator foramen. It will be appreciated that other anatomical landmarks or features can be segmented based on different input images and training data sets.

Thus, using the systems and methods described in the present disclosure, DDPM generative models can be used as powerful feature extractors to generate training data sets for few-shot image segmentation. A pre-trained DDPM, or other generative model, can be used to facilitate segmentation projects in medical image analysis and lower the costs associated with annotation of a large number of images.

An important aspect of preoperative planning in total hip arthroplasty (“THA”) and other orthopedic procedures is an appropriate implant selection and ideal positioning of these implants. Careful planning can reduce the risk of postoperative complications and improve patient outcomes.

Preoperative digital radiographs are routinely obtained before THA and other orthopedic procedures. It is an aspect of the present disclosure that a deep learning model (e.g., a generative model) can be used to assist with preoperative planning of THA, or other orthopedic or surgical procedure, candidate patients by generating synthetic inpainted images that depict realistic postoperative radiographs of those patients given a single preoperative radiograph.

In an example implementation, a total of 38,375 pairs of preoperative and postoperative pelvis radiographs were collected from 3,498 patients who underwent THA, but had not experienced a significant complication in two years following THA. A deep learning pipeline was developed based on a DDPM generative model to receive a preoperative pelvis radiograph, localize the hip joint with bounding boxes using a pre-trained deep learning model (e.g., a pre-trained YOLOv5 deep learning model), fill the identified bounding box with random Gaussian noise, and gradually denoise (inpaint) the noisy area to generate a virtual postoperative radiograph for the same patient. In the context of deep learning, a bounding box is a rectangular box (or other suitable boundary) used to identify the location and size of an object within an image. It is typically used in object detection models where the model learns to fit a bounding box around some objects of interest. Model outputs were compared to real postoperative radiographs. Furthermore, inference on a single input was made multiple times with different input noise values to yield additional virtual radiographs to demonstrate model confidence in its surgical choices.

In an example use, the deep learning algorithm took less than one minute to generate a single postoperative radiograph from an input preoperative radiograph. Implant appearance and positioning in the virtual outputs generated by the model were visually consistent with the real radiographs. Multiple inference runs for a single preoperative radiograph could be run to yield more than one virtual output with similar or different implant choices concerning the properties of stems, heads, cups, collars, and screws.

The deep learning algorithm can be used to help surgeons inspect virtual postoperative radiographs of THA candidate patients. Visualizing multiple virtual surgeries on the same input can inform users of model confidence in its surgical choices. The proposed tool may potentiate next-generation preoperative planning for THA, or other orthopedic or surgical procedures, while also increasing patient understanding of the procedure and facilitating the teaching of THA to orthopedic surgery residents.

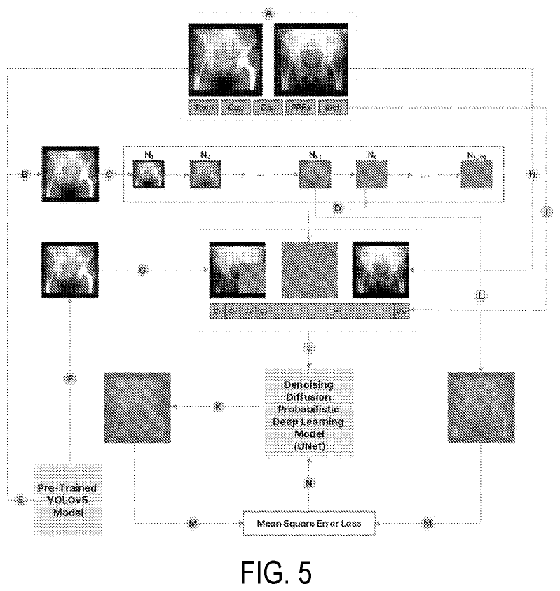

A schematic presentation of the training pipeline for the DDPM used for generating an inpainted medical image is depicted in FIG. 5. Each iteration of training includes multiple steps. At step A: An input pair of real preoperative and post-operative radiographs are loaded. At step B: The post-op radiograph is pre-processed and augmented. As a non-limiting example, data augmentation may include random horizontal flipping and/or random rotation over a range of degrees (e.g., a range of 10 degrees). At step C: Random Gaussian noise is added to the post-op radiograph for 1000 steps and using a pre-defined noise schedule. At step D: A random t value is sampled from 1 to 1000, and the noisy version of the post-operative radiograph at step t is selected. At step E: another instance of the postoperative radiograph is fed through a pre-trained YOLOv5 deep learning model, or other object detection model, to identify a target hip joint (e.g., one of the non-operated hip joints) on the image using a bounding box, as indicated at step F. At step G: The identified bounding box of the post-operative radiograph is filled with random Gaussian noise. At step H: The pre-operative radiograph is also pre-processed and augmented, similar to the post-operative radiograph. At step I: non-imaging variables (e.g., hardware choices) are one-hot encoded and concatenated together to form a one dimensional class vector. At step J: A three-channel image is built by concatenating the outputs of steps D, G, and I. This image is fed to the DDPM as its input. At step K: DDPM tries to predict the noisy version of the post-operative radiograph in step t−1 (as explained in step C). At step L: The real noisy version of the post-operative radiograph in step t−1 is selected and pre-processed. At step M: The difference between outputs of steps K and L is calculated using the mean square error loss function. At step N: The weights of the DDPM model are updated based on the gradients of the loss obtained in step L. The entire training continues for 170,000 iterations. During training, the DDPM model has been conditioned on the preoperative radiograph and a cropped post-operative radiograph (resembling a cropped preoperative radiograph in real case scenarios).

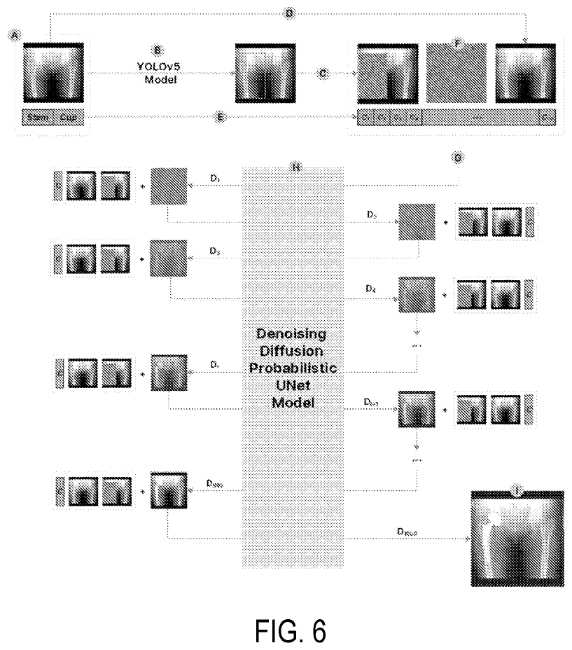

A schematic presentation of the inference pipeline for DDPM, or other generative model, used for generating an inpainted medical image is depicted in FIG. 6. At step A: a single preoperative input radiograph is loaded and pre-processed. At step B: A YOLOv5 deep learning model, or other object detection model, is used to detect a target hip joint (e.g., one of the non-operated hip joints) on the radiograph using a bounding box. At step C: The bounding box in the previous step is filled with noise. At step D: A separate intact copy of the pre-operative radiograph is also loaded and pre-processed. At step E: the hardware choices and several other non-imaging constants may be encoded and concatenated together to form a one-dimensional class vector (represented with “C”). At step F: A random tensor of Gaussian noise is generated with the same shape as the input radiograph. At step G: The combination of the outputs from steps C, D, E, and F are concatenated to form the DDPM inputs. At step H: The DDPM denoises its inputs for 1000 steps, where each denoising step receives the outputs of the previous step. At step I: The final virtual (synthetic) inpainted post-operative radiograph is obtained after the 1000th iteration of denoising. No postoperative radiographs are needed during inference as the model inputs.

In the illustrated embodiment, a trained version of the generative model may receive 2-4 distinct inputs from the user: the preoperative medical image (e.g., a preoperative anteroposterior (AP) pelvis radiograph), the target joint laterality (e.g., the target hip joint laterality), and optionally the desired hardware components (e.g., desired femoral and acetabular components, which may be selectable from a list of 25 femoral and 10 acetabular varieties, as an example). If the surgeon does not define any implant types, the model can operate in an unconditioned fashion (i.e., an Automated mode). Alternatively, implants can be designated, in which case the model will use them in its subsequent generations (i.e., a Hardware-aware mode). Upon receiving these inputs, the joint on the specified side will be detected by a pretrained model, and the preprocessed inputs will be fed to the DDPM model in 1000 steps (or another suitable number of steps). At each step, the image is gradually denoised to build a realistic postoperative radiograph, as described above.

The training and inference pipelines described above differ the preprocessing of the input data. During the inference, multiple preprocessing steps will take place on the input data: 1) a previously trained object detector DL model (e.g., YOLOv5) fits a bounding box around the hip joint in the desired side; 2) A three-channel tensor is built, including the designated joint of interest, the original preoperative radiograph, and a tensor completely filled with random Gaussian noise; and 3) the hardware choices of the surgeon are one-hot encoded and concatenated with several other constants to form a one-dimensional class vector (e.g., of size 50).

The preceding preprocessed data are combined and fed to the DDPM network in 1000 steps, as an example. At each step, the image is gradually denoised to build a realistic postoperative radiograph that matches the surrounding context of the preoperative radiograph and meets the hardware choices selected by the user, if specified.

During training, a similar three-channel input tensor is constructed, but with slightly different components. While two of the three channels will continue to be the input preoperative radiograph and the tensor filled with random Gaussian noise, the third channel instead includes a postoperative radiograph (instead of the preoperative radiograph) that is filled with noise in the joint area by the help of the object detector model. This enables the model to be trained using real patient data from existing registries, while the model will be optimized to generate a postoperative radiograph as close as possible to the patient's ground truth postoperative imaging.

Another distinction is in the preprocessing of non-imaging data. In addition to the ground truth stem and cup types, the medical image inpainting model may also receive inputs for the dislocation and PPFx outcomes of the patient, as well as the intended range of inclination angles it should use to generate postoperative radiographs. In some examples, the model expects one of three possible input codes for each THA complication: 1 (complication occurred within two years of surgery), 2 (no complications occurred within two years), or 0 (unknown two-year complication status). Similarly, the model may anticipate a binary code for the desired inclination angle:/(inclination angle between 27 and 47 degrees, which is the proposed safe zone in (IS)) and 2 (inclination angle less than 27 degrees or greater than 47 degrees). Using this engineering technique, the model may be trained on a wide variety of radiographs with diverse inclination angle selections and complication outcomes, thereby maximizing the variance of training data. During the inference, the model may be operated so that the generated radiographs have inclination angles between 27 and 47 degrees, and the patient does not have any complications after the model-performed synthetic THA. Consequently, the non-imaging variables that the user can modify during inference may be limited to stem and cup type specifications, and the encodings for complication outcomes and desirable inclination angles may be predefined to constant values (2 for complication outcomes and/for inclination angles).

In some implementations, the medical image inpainting model may randomly switch between automated and hardware-aware generations during the training. During the automated generations, the encodings for one or more non-imaging variables are randomly replaced with 0, signaling the model that there are no input specifications for those variables in that generation round. This switching strategy enables the model to function reliably in both hardware-aware and automated modes of generation.

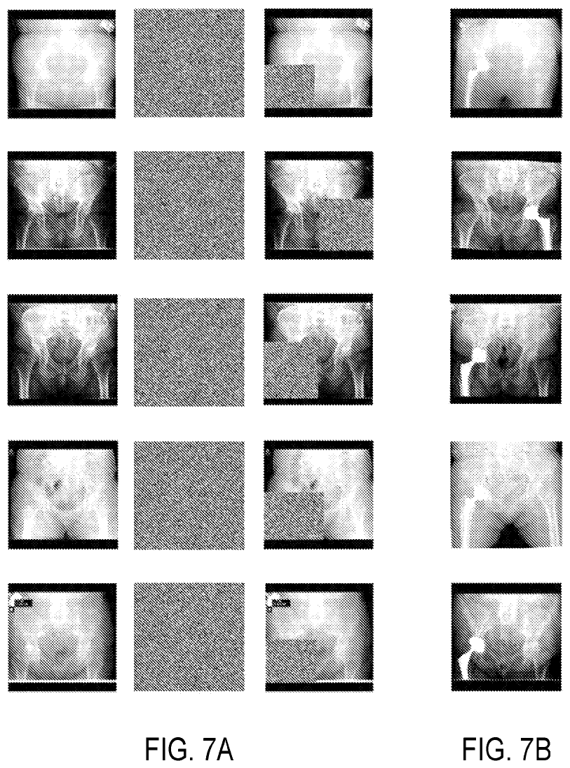

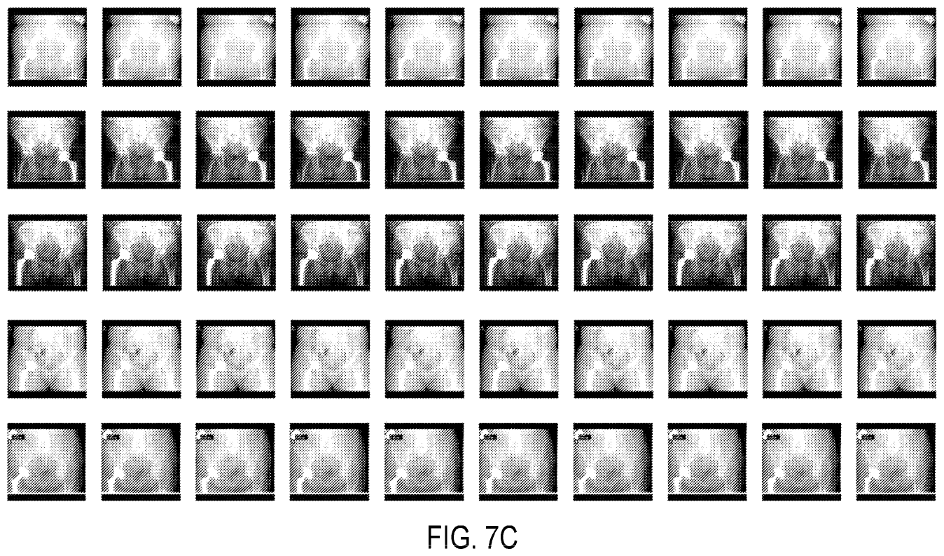

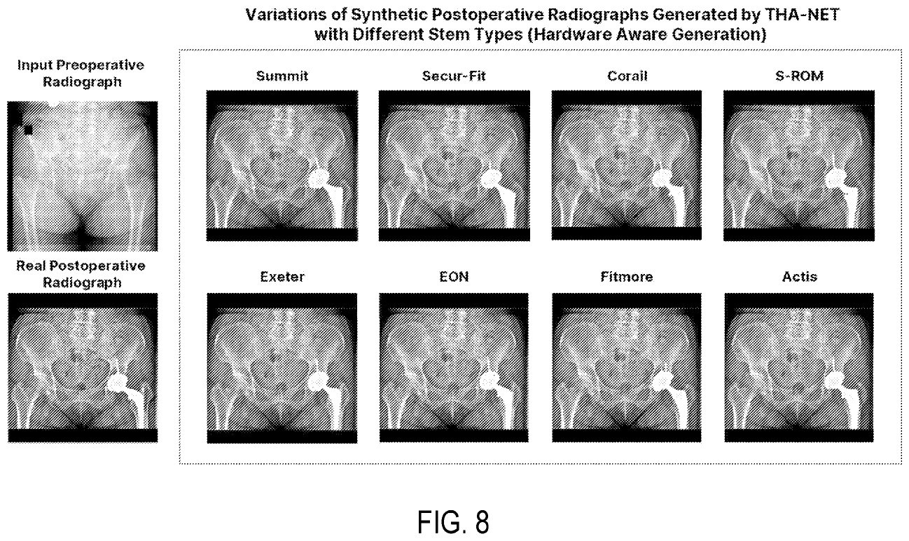

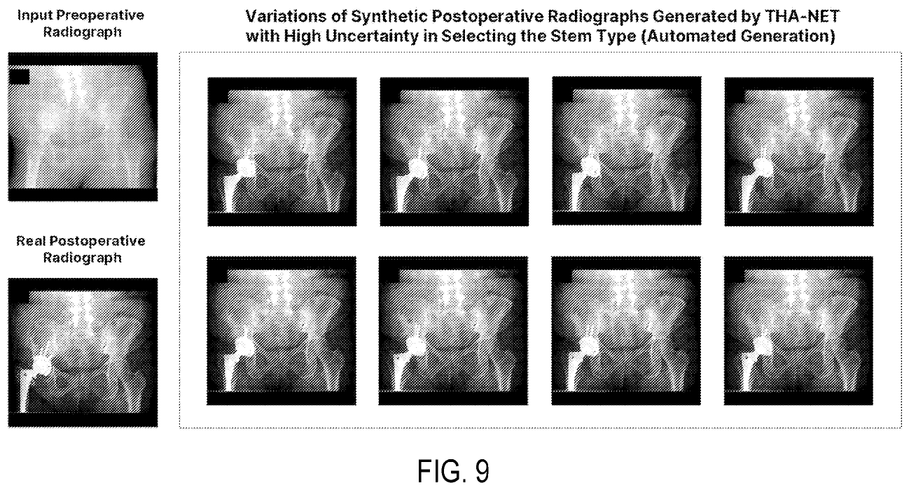

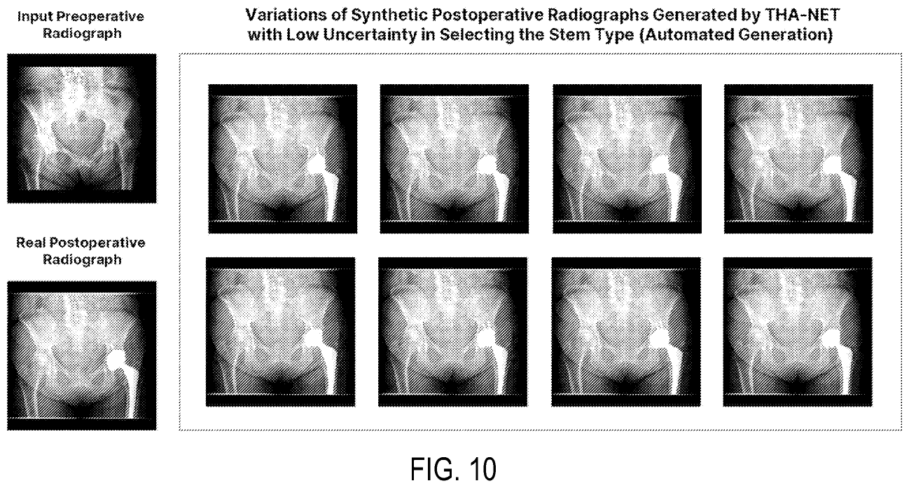

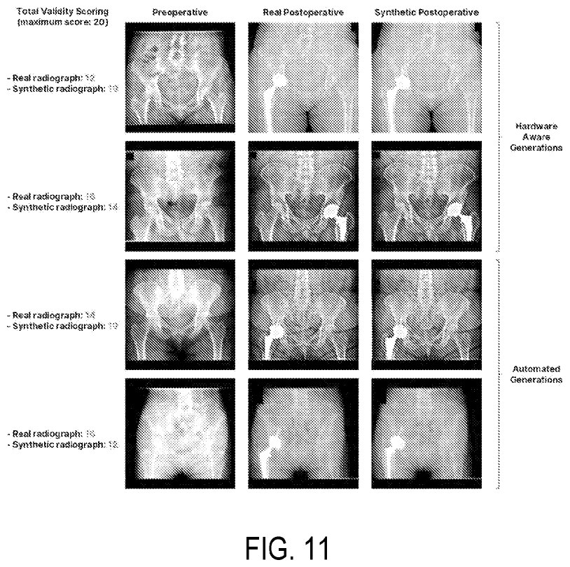

Examples for the real-case performance of the DDPM-based inpainting model are shown in FIGS. 7A and 7B. FIG. 7A shows inputs of the DDPM model, which for each example include an image with three channels: 1) a real preoperative radiograph, 2) the same preoperative radiograph but with random Gaussian noise added to the hip joint bounding box, and 3) a full tensor of random Gaussian noise with the same shape as the input radiograph. Generating the last channel with different values could yield multiple instances of virtual surgeries. FIG. 7B shows real postoperative radiographs for the patient. FIG. 7C shows example inpainted medical images generated using a DDPM trained as described above. FIG. 8 illustrates variations of synthetic postoperative radiographs generated by the disclosed generative models (referred to as THA-Net in the illustrated example) for a single preoperative radiograph with different prespecified femoral component types in each generation. The generations are done in the Hardware-aware mode, but the choice of acetabular component was not prespecified to the model. FIG. 9 illustrates variations of synthetic postoperative radiographs generated by THA-Net for a single preoperative radiograph within the Automated mode. THA-Net has used different femoral component types for generating different synthetic postoperative radiographs, a finding that may be related to the non-standard preoperative radiograph fed to the model during generation (top left hand side). FIG. 10 illustrates variations of synthetic postoperative radiographs generated by THA-Net for a single preoperative radiograph within the Automated mode. THA-Net has used the same femoral component type for generating different synthetic postoperative radiographs, a finding that indicates the certainty of the model in its hardware choices for performing the templating task. FIG. 11 illustrates visualizations of images from reviewer evaluation pools with highest and lowest total validity ratings in Hardware-aware and Automated generations. Each reviewer rated the validity of radiographs on a 10-point Likert scale, so the maximum possible rating is 20. The ratings of real counterparts of generated radiographs are also provided for comparison purposes.

As described above, templating is an important preoperative planning step for THA and other orthopedic procedures in order to anticipate and minimize complications. Recent technologies have made it easier for surgeons to achieve predefined templating targets, but fewer efforts have been made to aid surgeons in selecting the proper target. Using the systems and methods described in the present disclosure, a specialized DDPM generates synthetic postoperative radiographs as a templating tool to aid in identifying idealized patient-specific postoperative radiographic targets for THA and other orthopedic procedures.

The disclosed systems and methods enable next generation THA, and other orthopedic procedure, templating with AI-assisted ideal implant placement and surgical execution. On the one hand, a hardware-aware mode of the disclosed generative models provides surgeons the ability to prespecify implants in the synthetic radiographs. On the other hand, an automated mode shows the morphology of the ideal implant for the patient according to an interpretation of their anatomy and pathology. The surgeon also has the ability to run the model multiple times and observe if the model is consistent in its hardware choices (indicating high confidence in implant choice), or if the model generates radiographs with multiple implant types, suggesting some level of uncertainty. As noted above in a non-limiting example, the generative model may be differentially trained on patients that did and did not sustain postoperative dislocation and PPFx and had an acetabular inclination angle between 27-47 degrees. Thus, in this way, the model can be constructed to incorporate a goal to generate radiographs more likely to avoid these complications and hit the acetabular target.

The disclosed generative models demonstrate powerful properties to enable patient-specific surgical planning both with respect to implant selection as well as implant placement. This has important implications for selecting optimal postoperative targets, which has proven a more challenging endeavor compared to the question of whether any given target can be hit. The latter goal is reliably achieved with surgical experience, or increasingly with enabling technologies such as navigation, robotics, and AR/VR. The disclosed generative model can be trained on thousands of radiographs from real THAs, or other orthopedic procedures, and can be optimized to incorporate the amalgamation of such data, while preferentially generating features associated with good outcomes. Accordingly, the generated synthetic images for a patient account for a range of prior experiences.

Advantageously, the systems and methods described in the present disclosure may interface with robotics, navigation, and AR/VR technologies that are increasingly used to hit a desired target, while enabling this outcome in a way that does not require computed tomography scans.

It is another aspect of the present disclosure to provide systems and methods for detecting and removing radiographic markers from radiographs of different anatomical regions, yielding images that are anonymized. In general, a deep learning model can be trained and implemented to detect and remove such radiographic markers. As a non-limiting example, a two-pass approach to localize and characterize radiologic markers may, which is fast and reliable for image anonymization, may be used. Fine tuning a localizer network can increase deidentification performance. In some implementations, selective retention of markers can be used and may enable granular control over image deidentification.

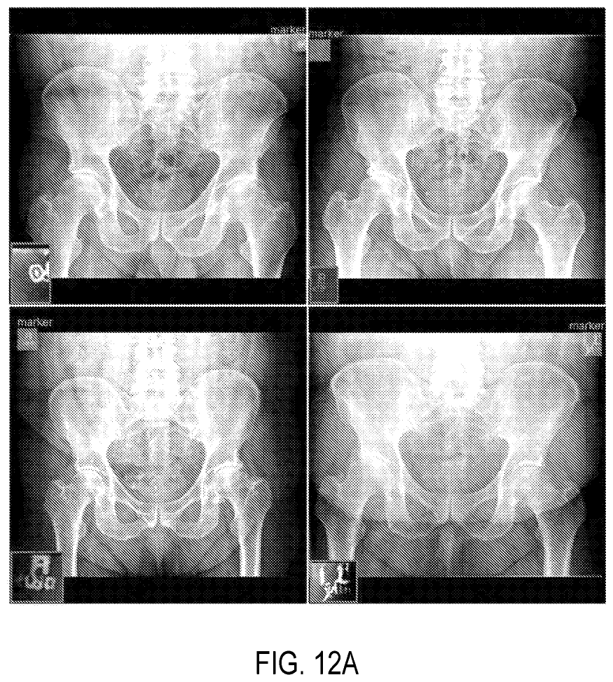

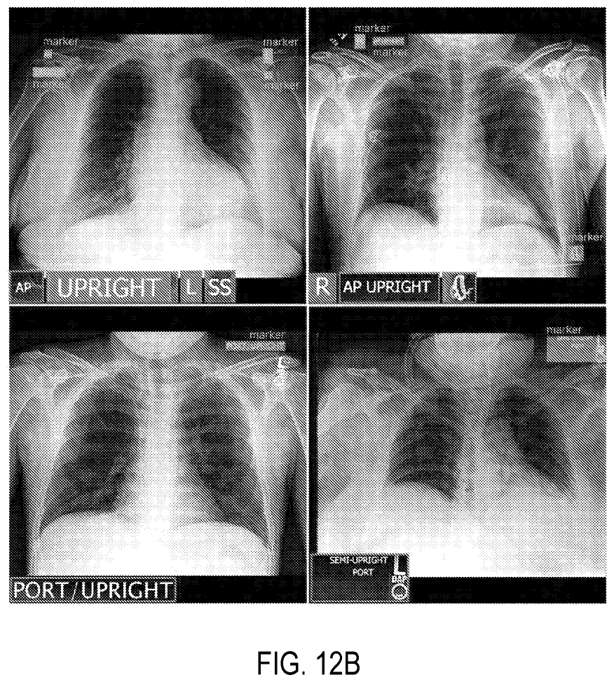

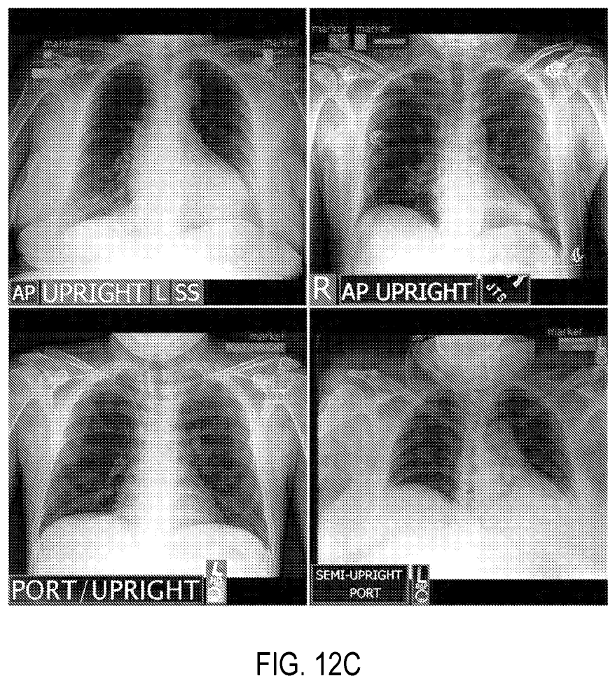

Radiographic markers contain protected health information (PHI) and their removal is therefore generally needed before public release of medical images containing those markers. As noted above, the systems and methods described in the present disclosure provide a deep learning (DL) model that localizes radiographic markers and selectively removes them to enable deidentified data sharing. FIGS. 12A-12C illustrate examples of medical images processed to identify and localize radiographic markers for redaction. The identified radiographic markers are shown as enlarged in the insets in each image.

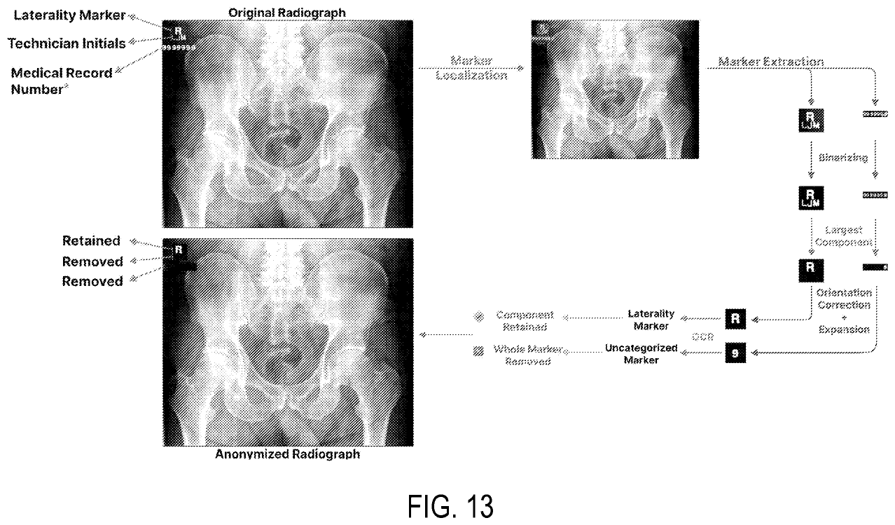

Although removing identification markers is a desired outcome, it may be advantageous in some instances to retain some markers for preprocessing steps. For example, laterality markers in medical image data may be retained. A dynamic threshold can be used to binarize the marker area, and then the largest connected component was selected. This component is sent to an algorithm that automatically corrects the marker's orientation to make it upright. Finally, the marker is passed to an optical character recognition (OCR) algorithm that utilizes a long short-term memory (LSTM) to detect isolated “R” and “L” characters on the image. If the component is detected as either of these characters, it will be retained in the original image; otherwise, it will be replaced by black pixels (i.e., zeroed pixel values). An example workflow of this process is illustrated in FIG. 13.

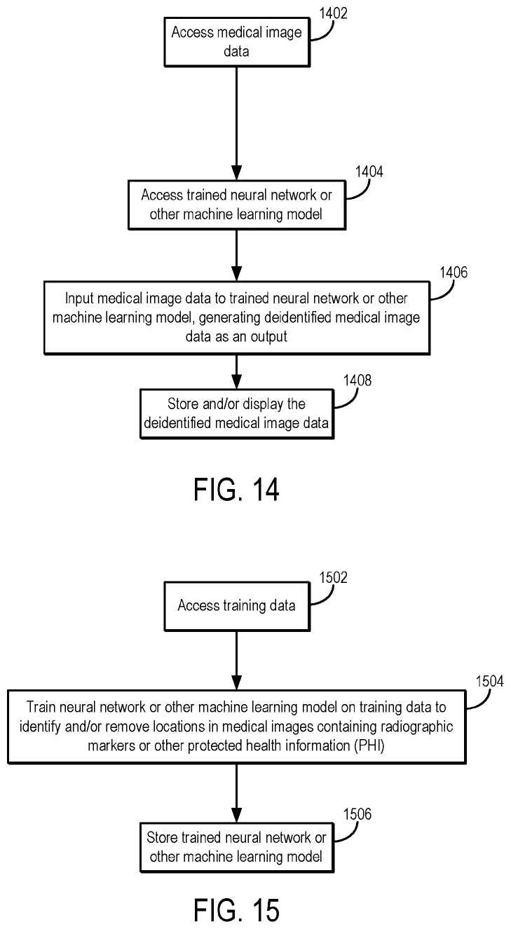

Referring now to FIG. 14, a flowchart is illustrated as setting forth the steps of an example method for generating deidentified medical image data using a suitably trained neural network or other machine learning algorithm. As will be described, the neural network or other machine learning algorithm takes medical image data as input data and generates deidentified medical image data as output data. As an example, the deidentified medical image data includes medical images that have been deidentified by removing radiographic markers and other PHI from the images.

The method includes accessing medical image data with a computer system, as indicated at step 1402. Accessing the medical image data may include retrieving such data from a memory or other suitable data storage device or medium. Additionally or alternatively, accessing the medical image data may include acquiring such data with a medical imaging system and transferring or otherwise communicating the data to the computer system, which may be a part of the medical imaging system.

In general, the medical image data may include medical images having one or more radiographic markers. The radiographic markers may include, for example, markers present in the medical images that indicate the patient name, type of exam, institution, and staff. Additionally, the radiographic markers may include other text or markers in the medical images that include PHI.

A trained neural network (or other suitable machine learning algorithm) is then accessed with the computer system, as indicated at step 1404. In general, the neural network is trained, or has been trained, on training data in order to locate and remove radiographic markers or other PHI in medical images.

Accessing the trained neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed.

An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value; an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer. Similar to the input layer, the output layer typically has the same number of nodes as the possible outputs. In an example in which the artificial neural network generates deidentified medical image data, the output layer may include nodes that output deidentified medical images and/or locations in the medical images where radiographic markers have been located.

The medical image data are then input to the one or more trained neural networks, generating output as deidentified medical image data, as indicated at step 1406. As an example, the deidentified medical image data may include medical images in which one or more radiographic markers have been located and removed. For instance, radiographic markers can be located as replaced with empty pixels (e.g., zero-valued pixels), random noise, or the like. In some instances, the located radiographic markers may be inpainted using the inpainting techniques described in the present disclosure.

The deidentified medical image data generated by inputting the medical image data to the trained neural network(s) can then be displayed to a user, stored for later use or further processing, or both, as indicated at step 1408. In some instances, the deidentified medical image data generated by the machine learning model may include medical images with radiographic markers identified, but not removed. In these instances, the deidentified medical image data may be further processed to remove the identified locations corresponding to the radiographic markers or other PHI. For instance, the radiographic markers may be manually or automatically redacted (e.g., by zeroing the pixel values, by randomizing the pixel values). In some other instances, the deidentified medical image data may be further processed using an inpainting technique to inpaint the identified locations of the medical images that contain radiographic markers or other PHI.

As a non-limiting example, in some implementations a two-pass approach is taken. In the first pass, the machine learning model detects all marker areas to prevent PHI leak. In the second pass, an OCR algorithm is used to allow informative tags, like the laterality markers, to bypass redaction. This modular approach also facilitates adding filters to find and stop the removal of certain markers, like those that are specific to an institution. Moreover, due to the large variability between different institutions in the types and use cases of imaging markers, having an easily adaptable approach that can be finetuned with minimal effort is desirable. The second pass can be disabled while training a DL model to prevent shortcut learning based on burnt-in markers.

Referring now to FIG. 15, a flowchart is illustrated as setting forth the steps of an example method for training one or more neural networks (or other suitable machine learning algorithms) on training data, such that the one or more neural networks are trained to receive medical image data as input data in order to generate deidentified medical image data as output data, where the deidentified medical image data include medical images in which radiographic markers or other PHI have been located and removed.

In general, the neural network(s) can implement any number of different neural network architectures. For instance, the neural network(s) could implement a convolutional neural network, a residual neural network, or the like. Alternatively, the neural network(s) could be replaced with other suitable machine learning or artificial intelligence algorithms, such as those based on supervised learning, unsupervised learning, deep learning, ensemble learning, dimensionality reduction, and so on. As a non-limiting example, the deidentification model may implement a YOLOv5 object detection framework.

The method includes accessing training data with a computer system, as indicated at step 1502. Accessing the training data may include retrieving such data from a memory or other suitable data storage device or medium. Alternatively, accessing the training data may include acquiring such data with a medical imaging system and transferring or otherwise communicating the data to the computer system.

In general, the training data can include medical images, such as radiographs, computed tomography (CT) images, magnetic resonance images, ultrasound images, positron emission tomography (PET) images, optical images, and so on. In some embodiments, the training data may include medical images that have been labeled (e.g., labeled as containing patterns, features, or characteristics indicative of radiographic markers; and the like).

The method can include assembling training data from medical images using a computer system. This step may include assembling the medical images into an appropriate data structure on which the neural network or other machine learning algorithm can be trained. Assembling the training data may include assembling medical images, segmented medical images, and other relevant data. For instance, assembling the training data may include generating labeled data and including the labeled data in the training data. Labeled data may include medical images, segmented medical images, or other relevant data that have been labeled as belonging to, or otherwise being associated with, one or more different classifications or categories. For instance, labeled data may include medical images and/or segmented medical images (or patches thereof) that have been labeled as being associated with one or more radiographic markers or PHI.

As a non-limiting example, 2000 hip and pelvic radiographs were annotated to train a YOLOv5-x model. Data was split into training, validation, and test sets at the patient level. Extracted markers were then characterized using an image processing algorithm, and potentially useful markers (e.g. ‘L’ and ‘R’) without identifying information were retained. All markers that were related to the patient, type of exam, institution, and staff were annotated. As an example, the markers may be annotated by drawing a bounding box around them. Then, the data were split at the patient level into 60%/20%/20% for training, validation, testing, respectively. Images were resampled to 512×512 pixels and a YOLOv5-x model was trained to detect the marker location.

One or more neural networks (or other suitable machine learning algorithms) are trained on the training data, as indicated at step 1504. In general, the neural network can be trained by optimizing network parameters (e.g., weights, biases, or both) based on minimizing a loss function. As one non-limiting example, the loss function may be a mean squared error loss function.

Training a neural network may include initializing the neural network, such as by computing, estimating, or otherwise selecting initial network parameters (e.g., weights, biases, or both). During training, an artificial neural network receives the inputs for a training example and generates an output using the bias for each node, and the connections between each node and the corresponding weights. For instance, training data can be input to the initialized neural network, generating output as deidentified medical image data. The artificial neural network then compares the generated output with the actual output of the training example in order to evaluate the quality of the deidentified medical image data. For instance, the deidentified medical image data can be passed to a loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current neural network and its associated network parameters represent the trained neural network. Different types of training processes can be used to adjust the bias values and the weights of the node connections based on the training examples. The training processes may include, for example, gradient descent, Newton's method, conjugate gradient, quasi-Newton, Levenberg-Marquardt, among others.

The artificial neural network can be constructed or otherwise trained based on training data using one or more different learning techniques, such as supervised learning, unsupervised learning, reinforcement learning, ensemble learning, active learning, transfer learning, or other suitable learning techniques for neural networks. As an example, supervised learning involves presenting a computer system with example inputs and their actual outputs (e.g., categorizations). In these instances, the artificial neural network is configured to learn a general rule or model that maps the inputs to the outputs based on the provided example input-output pairs.

As a non-limiting example, a deidentification model may be trained for 500 epochs with a batch size of 8 and an image size of 512 pixels. The initial learning rate may be set to 0.01. Standard data augmentations such as random cropping, flipping, and color jittering may also be used to improve the robustness of the model. The validation set may be used to select the confidence threshold that maximizes precision. For the intersection over union (IoU) threshold, a grid search on the validation set can be performed to maximize precision of the model.

The model may be finetuned on a smaller set of medical images (e.g., 20 chest radiographs) using the same model architecture (e.g., the same YOLOv5 object detection framework). Weights from a previous step can be used as the basis of finetuning. Additionally, the smaller set of medical images used for fine tuning may be oversampled, such as by a factor of 50. In an example, the model was fine tuned for 50 epochs with a batch size of 8 and an image size of 512 pixels. The initial learning rate was set to 0.005. The same data augmentations used in the initial training may be used when fine tuning the model. The fine tuning configuration may include the same hyperparameters as in the initial training. In some instances, a first number of layers (e.g., the first nine layers) of the model may be frozen during fine tuning to preserve the learned features from the initial training. Fine Tuning allows for the model to adapt to a new domain of medical images (e.g., chest radiographs versus pelvis radiographs used for training the initial model) and improve its performance on such an external dataset. By fine tuning on a small number of images from the target domain, a pretrained model can be readily adapted to new datasets and imaging modalities.

The one or more trained neural networks are then stored for later use, as indicated at step 1506. Storing the neural network(s) may include storing network parameters (e.g., weights, biases, or both), which have been computed or otherwise estimated by training the neural network(s) on the training data. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

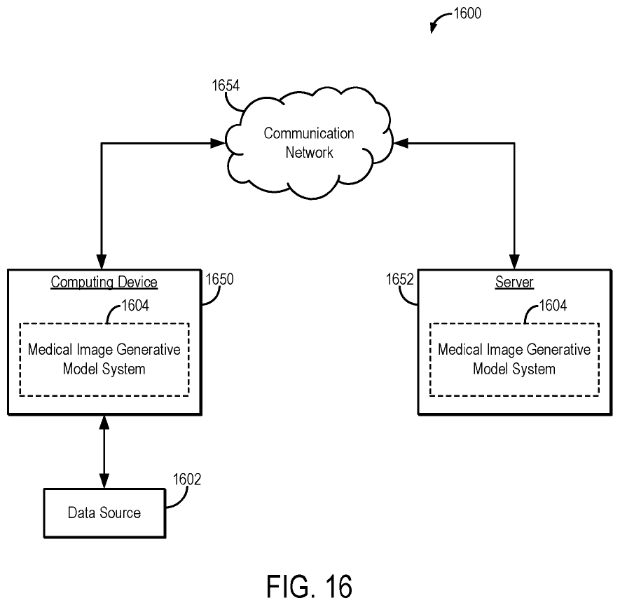

Referring now to FIG. 16, an example of a system 1600 for generative modelling of medical images in accordance with some embodiments of the systems and methods described in the present disclosure is shown. As shown in FIG. 16, a computing device 1650 can receive one or more types of data (e.g., digital radiograph data, medical image data, patient health data) from data source 1602. In some embodiments, computing device 1650 can execute at least a portion of a medical image generative model system 1604 to generate synthetic medical images, segmented medical images, inpainted medical images, and/or deidentified medical images from data received from the data source 1602 using one or more generative models.

Additionally or alternatively, in some embodiments, the computing device 1650 can communicate information about data received from the data source 1602 to a server 1652 over a communication network 1654, which can execute at least a portion of the medical image generative model system 1604. In such embodiments, the server 1652 can return information to the computing device 1650 (and/or any other suitable computing device) indicative of an output of the medical image generative model system 1604.

In some embodiments, computing device 1650 and/or server 1652 can be any suitable computing device or combination of devices, such as a desktop computer, a laptop computer, a smartphone, a tablet computer, a wearable computer, a server computer, a virtual machine being executed by a physical computing device, and so on. The computing device 1650 and/or server 1652 can also reconstruct images from the data.

In some embodiments, data source 1602 can be any suitable source of data (e.g., measurement data, images reconstructed from measurement data, processed image data), such as a medical imaging system (e.g., a digital radiography system, an x-ray fluoroscopy system, a CT system, an MRI system, an ultrasound system), another computing device (e.g., a server storing measurement data, images reconstructed from measurement data, processed image data), and so on. In some embodiments, data source 1602 can be local to computing device 1650. For example, data source 1602 can be incorporated with computing device 1650 (e.g., computing device 1650 can be configured as part of a device for measuring, recording, estimating, acquiring, or otherwise collecting or storing data). As another example, data source 1602 can be connected to computing device 1650 by a cable, a direct wireless link, and so on. Additionally or alternatively, in some embodiments, data source 1602 can be located locally and/or remotely from computing device 1650, and can communicate data to computing device 1650 (and/or server 1652) via a communication network (e.g., communication network 1654).

In some embodiments, communication network 1654 can be any suitable communication network or combination of communication networks. For example, communication network 1654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 1654 can be a local area network, a wide area network, a public network (e.g., the Internet), a private or semi-private network (e.g., a corporate or university intranet), any other suitable type of network, or any suitable combination of networks. Communications links shown in FIG. 16 can each be any suitable communications link or combination of communications links, such as wired links, fiber optic links, Wi-Fi links, Bluetooth links, cellular links, and so on.

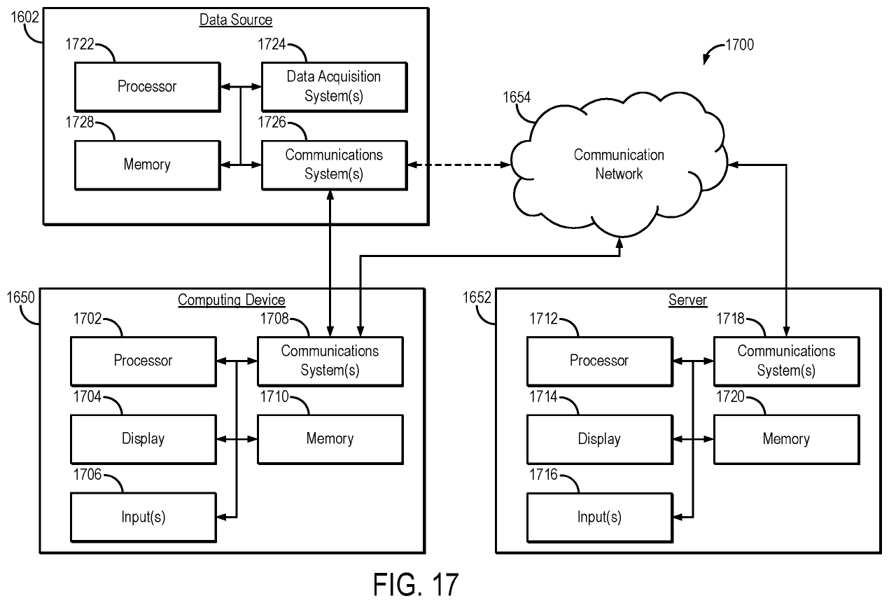

Referring now to FIG. 17, an example of hardware 1700 that can be used to implement data source 1602, computing device 1650, and server 1652 in accordance with some embodiments of the systems and methods described in the present disclosure is shown.

As shown in FIG. 17, in some embodiments, computing device 1650 can include a processor 1702, a display 1704, one or more inputs 1706, one or more communication systems 1708, and/or memory 1710. In some embodiments, processor 1702 can be any suitable hardware processor or combination of processors, such as a central processing unit (“CPU”), a graphics processing unit (“GPU”), and so on. In some embodiments, display 1704 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 1706 can include any suitable input devices and/or sensors that can be used to receive user input, such as a keyboard, a mouse, a touchscreen, a microphone, and so on.

In some embodiments, communications systems 1708 can include any suitable hardware, firmware, and/or software for communicating information over communication network 1654 and/or any other suitable communication networks. For example, communications systems 1708 can include one or more transceivers, one or more communication chips and/or chip sets, and so on. In a more particular example, communications systems 1708 can include hardware, firmware, and/or software that can be used to establish a Wi-Fi connection, a Bluetooth connection, a cellular connection, an Ethernet connection, and so on.

In some embodiments, memory 1710 can include any suitable storage device or devices that can be used to store instructions, values, data, or the like, that can be used, for example, by processor 1702 to present content using display 1704, to communicate with server 1652 via communications system(s) 1708, and so on. Memory 1710 can include any suitable volatile memory, non-volatile memory, storage, or any suitable combination thereof. For example, memory 1710 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 1710 can have encoded thereon, or otherwise stored therein, a computer program for controlling operation of computing device 1650. In such embodiments, processor 1702 can execute at least a portion of the computer program to present content (e.g., images, user interfaces, graphics, tables), receive content from server 1652, transmit information to server 1652, and so on. For example, the processor 1702 and the memory 1710 can be configured to perform the methods described herein (e.g., the synthetic image generation methods described above, the feature extraction and image segmentation methods described above, the inpainting methods described above, the medical image deidentification methods described above).

In some embodiments, server 1652 can include a processor 1712, a display 1714, one or more inputs 1716, one or more communications systems 1718, and/or memory 1720. In some embodiments, processor 1712 can be any suitable hardware processor or combination of processors, such as a CPU, a GPU, and so on. In some embodiments, display 1714 can include any suitable display devices, such as an LCD screen, LED display, OLED display, electrophoretic display, a computer monitor, a touchscreen, a television, and so on. In some embodiments, inputs 1716 can include any suitable input devices and/or sensors that can be used to receive user input, such as a keyboard, a mouse, a touchscreen, a microphone, and so on.

In some embodiments, communications systems 1718 can include any suitable hardware, firmware, and/or software for communicating information over communication network 1654 and/or any other suitable communication networks. For example, communications systems 1718 can include one or more transceivers, one or more communication chips and/or chip sets, and so on. In a more particular example, communications systems 1718 can include hardware, firmware, and/or software that can be used to establish a Wi-Fi connection, a Bluetooth connection, a cellular connection, an Ethernet connection, and so on.

In some embodiments, memory 1720 can include any suitable storage device or devices that can be used to store instructions, values, data, or the like, that can be used, for example, by processor 1712 to present content using display 1714, to communicate with one or more computing devices 1650, and so on. Memory 1720 can include any suitable volatile memory, non-volatile memory, storage, or any suitable combination thereof. For example, memory 1720 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1720 can have encoded thereon a server program for controlling operation of server 1652. In such embodiments, processor 1712 can execute at least a portion of the server program to transmit information and/or content (e.g., data, images, a user interface) to one or more computing devices 1650, receive information and/or content from one or more computing devices 1650, receive instructions from one or more devices (e.g., a personal computer, a laptop computer, a tablet computer, a smartphone), and so on.

In some embodiments, the server 1652 is configured to perform the methods described in the present disclosure. For example, the processor 1712 and memory 1720 can be configured to perform the methods described herein (e.g., the synthetic image generation methods described above, the feature extraction and image segmentation methods described above, the inpainting methods described above, the medical image deidentification methods described above).

In some embodiments, data source 1602 can include a processor 1722, one or more data acquisition systems 1724, one or more communications systems 1726, and/or memory 1728. In some embodiments, processor 1722 can be any suitable hardware processor or combination of processors, such as a CPU, a GPU, and so on. In some embodiments, the one or more data acquisition systems 1724 are generally configured to acquire data, images, or both, and can include a medical imaging system (e.g., a digital radiography system, an x-ray fluoroscopy system, a CT system, an MRI system, an ultrasound system). Additionally or alternatively, in some embodiments, the one or more data acquisition systems 1724 can include any suitable hardware, firmware, and/or software for coupling to and/or controlling operations of a medical imaging system. In some embodiments, one or more portions of the data acquisition system(s) 1724 can be removable and/or replaceable.

Note that, although not shown, data source 1602 can include any suitable inputs and/or outputs. For example, data source 1602 can include input devices and/or sensors that can be used to receive user input, such as a keyboard, a mouse, a touchscreen, a microphone, a trackpad, a trackball, and so on. As another example, data source 1602 can include any suitable display devices, such as an LCD screen, an LED display, an OLED display, an electrophoretic display, a computer monitor, a touchscreen, a television, etc., one or more speakers, and so on.

In some embodiments, communications systems 1726 can include any suitable hardware, firmware, and/or software for communicating information to computing device 1650 (and, in some embodiments, over communication network 1654 and/or any other suitable communication networks). For example, communications systems 1726 can include one or more transceivers, one or more communication chips and/or chip sets, and so on. In a more particular example, communications systems 1726 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1728 can include any suitable storage device or devices that can be used to store instructions, values, data, or the like, that can be used, for example, by processor 1722 to control the one or more data acquisition systems 1724, and/or receive data from the one or more data acquisition systems 1724; to generate images from data; present content (e.g., data, images, a user interface) using a display; communicate with one or more computing devices 1650; and so on. Memory 1728 can include any suitable volatile memory, non-volatile memory, storage, or any suitable combination thereof. For example, memory 1728 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1728 can have encoded thereon, or otherwise stored therein, a program for controlling operation of data source 1602. In such embodiments, processor 1722 can execute at least a portion of the program to generate images, transmit information and/or content (e.g., data, images, a user interface) to one or more computing devices 1650, receive information and/or content from one or more computing devices 1650, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for generating an inpainted medical image, comprising:
(a) accessing a preoperative medical image with a computer system, the preoperative medical image depicting patient anatomy;
(b) accessing a generative model with the computer system, the generative model having been trained on training data comprising preoperative and postoperative medical images; and
(c) inputting the preoperative medical image to the generative model using the computer system, generating an output as an inpainted medical image that depicts a predicted postoperative outcome for the patient.

2. The method of claim 1, wherein the generative model comprises a denoising diffusion probabilistic model (DDPM).

3. The method of claim 1, inputting the preoperative medical image to the generative model comprises:
selecting a region-of-interest in the preoperative medical image;
generating a partially augmented image by adding noise to the region-of-interest in the preoperative medical image;
inputting the partially augmented image to the generative model; and
wherein the inpainted medical image depicts the predicted postoperative outcome for the patient in the selected region-of-interest.

4. The method of claim 3, wherein selecting the region-of-interest comprises:
accessing an object detection model with the computer system; and
inputting the preoperative medical image to the object detection model using the computer system, generating an output as a bounding box identifying the region-of-interest in the preoperative medical image.

5. The method of claim 3, wherein the preoperative medical image and the partially augmented image are concatenated with a random noise image, and the concatenated images are input to the generative model.

6. The method of claim 1, wherein the preoperative medical image comprises a digital radiographic images.

7. The method of claim 6, wherein the predicted postoperative outcome comprises a position and type of medical implant for the patient.

8. The method of claim 7, wherein the medical implant comprises an orthopedic implant.

9. The method of claim 8, wherein the orthopedic implant comprises one of a hip implant, a knee implant, a shoulder implant, a spinal fixation implant, or a bone fixation implant.

10. The method of claim 7, wherein the generative model is conditioned on the type of the medical implant.

11. The method of claim 8, wherein the predicted postoperative outcome depicted in the inpainted medical image is indicative of the position and type of orthopedic implant that minimizes postoperative complications for the patient.

12. The method of claim 1, further comprising inputting patient health data to the generative model in addition to the preoperative medical image.

13. The method of claim 12, wherein the patient health data comprise at least one of a patient age, a patient biological sex, a patient ethnicity, a patient body mass index (BMI), a patient indication for surgery, a patient choice for surgical approach, or a choice of surgical implant type.

14. The method of claim 12, wherein the generative model is conditioned on the patient health data.

15. The method of claim 1, wherein the predicted postoperative outcome depicted in the inpainted medical image is indicative of a musculoskeletal reconstruction following trauma to the patient depicted in the preoperative medical image.

16. A method for generating synthetic medical images, comprising:
(a) accessing medical image data with a computer system, the medical image data depicting patient anatomy;
(b) accessing a generative model with the computer system, the generative model having been trained on training data comprising medical images consistent with the medical image data; and
(c) inputting the medical image data to the generative model using the computer system, generating an output as synthetic medical image data having an augmented feature.

17-29. (canceled)

30. A method for generating training data for use with training a machine learning model to generate segmented medical image data, comprising:
(a) accessing a pre-trained generative model with a computer system, the pre-trained generative model having been trained on training data comprising medical images;
(b) accessing medical image data with the computer system, the medical image data being consistent with the training data;
(c) inputting the medical image data to the generative model using the computer system;
(d) extracting, by the computer system, feature data from the generative model while the generative model is processing the medical image data; and
(e) training an image segmentation model using the feature data extracted from the generative model as a training data set.

31-35. (canceled)

36. A method for generating a deidentified medical image, the method comprising:
(a) accessing a medical image with a computer system, wherein the medical image depicts a subject and at least one radiographic marker;
(b) accessing a machine learning model with the computer system, wherein the machine learning model has been trained on training data to identify and remove radiographic markers in medical image data;
(c) inputting the medical image to the machine learning model using the computer system, generating a deidentified medical image as an output, wherein the deidentified medical image has the at least one radiographic marker removed therefrom; and
(d) displaying the deidentified medical image to a user via the computer system.

37-44. (canceled)
